(19) United States
(12) Patent Application Publication
(10) Pub. No.: US 2021/0187008 A1
(43) Pub. Date: Jun. 24, 2021

(54) USE OF ACANTHOPANAX TRIFOLIATUS POLYSACCHARIDE ATP1-1 IN PREPARATION OF DRUGS FOR TREATMENT OF DIABETES

(71) Applicant: GUANGDONG PHARMACEUTICAL UNIVERSITY, Guangzhou (CN)

(72) Inventor: Yufang PAN, Guangzhou (CN)

(73) Assignee: GUANGDONG PHARMACEUTICAL UNIVERSITY, Guangzhou (CN)

(21) Appl. No.: 17/262,774

(22) PCT Filed: Jan. 15, 2020
(86) PCT No.: PCT/CN2020/072110

§ 371 Date: Jan. 25, 2021

(30) Foreign Application Priority Data
Aug. 1, 2019 (CN) 201910707095.3

(51) Int. Cl. A61K 31/715; A61P 3/10

(52) U.S. Cl. CPC A61K 31/715; A61P 3/10

(57) ABSTRACT

A method of treating diabetes includes the step of preparing Acanthopanax trifoliatus polysaccharide ATP1-1 into drugs for treating the diabetes. The Acanthopanax trifoliatus polysaccharide ATP1-1 can alleviate the symptoms of weight loss in diabetic mice and facilitate the weight increase of mice; ATP1-1 has a hypoglycemic effect on the diabetic mice, and can enhance the ability to control blood glucose, reduce blood glucose fluctuation, and achieve the effect of treating diabetes; ATP1-1 can effectively reverse the decrease of insulin caused by injury of pancreatic islet; ATP1-1 has various degrees of therapeutic effects on mice pancreatic islets and can inhibit apoptosis of pancreatic islet cells; ATP1-1 can effectively repair injury of a spleen and regulate the body's immunologic function.

### CROSS REFERENCE TO THE RELATED APPLICATIONS

This application is a national phase entry of International Application No. PCT/CN2020/072110, filed on Jan. 15, 2020, which is based upon and claims priority to Chinese Patent Application No. 201910707095.3, filed on Aug. 1, 2019, both of which are incorporated herein by reference in their entireties.

### TECHNICAL FIELD

The present invention relates to a new use of Acanthopanax trifoliatus polysaccharide ATP1-1, particularly to use of Acanthopanax trifoliatus polysaccharide ATP1-1 in preparation of drugs for treatment of diabetes.

### BACKGROUND

At present, diabetes has become the third chronic disease that seriously threatens human health, ranking behind tumors and cardiovascular diseases. Diabetes is a growing problem in both developed and developing countries. It causes serious and costly consequences, including blindness, heart disease, kidney disease, and complications caused by diabetes. According to estimation from the International Diabetes Federation, the number of diabetes patients in China will exceed 50 million in the next 20 years. Nowadays, the diabetes has a tendency to expand and become younger, and how to prevent and treat diabetes has become a major issue that the medical field pays attention to. The mechanisms of action of the currently popularized diabetes-treating drugs in the market include: 1) stimulating pancreatic islet β-cells to secrete insulin; 2) reducing intestinal absorption of glucose; 3) inhibiting glycogen production; 4) increasing the sensitivity of peripheral tissues to insulin. Additionally, existing diabetes treatment drugs are still mainly western medicines, and the common disadvantages of western medicines are drug resistance and side effects. At the same time, the existing western medicines for diabetes treatment in the market which have less side effects usually have higher price.

Acanthopanax trifoliatus (Linn.) Merr. is a climbing shrub, which belongs to Acanthopanax of Arahaceae, having the effects of clearing away heat and toxic materials, dispelling wind and eliminating dampness, eliminating blood stasis and relieving pain, and nourishing middle warmer and tonifying Qi. In the preliminary research of inventors, the crude polysaccharide ATP (Acanthopanax trifoliatus polysaccharide) was prepared from the stems of Acanthopanax trifoliatus by water extraction and alcohol precipitation, the neutral polysaccharide ATP1 and the acidic polysaccharose ATP2 and ATP3 were separated by DEAE-52 cellulose column chromatography. ATP1 underwent repeatedly Sephadex G-75 dextran gel column chromatography to obtain neutral polysaccharide ATP1-1 with uniform molecular weight, however, the previous study did not disclose the effect of ATP1-1 on diabetes. The invention with the application number CN2013100356079 discloses the use of Acanthopanax trifoliatus polysaccharide in preparation of drugs for treatment of diabetes; however, it is well known that, at that time, the extraction method of polysaccharides from the Acanthopanax trifoliatus was relatively traditional, wherein the polysaccharide contained a lot of impurities such as nucleic acids, proteins and peptides, and the product has low purity, which was a mixture of multiple components. Therefore, under the technical background at that time, the polysaccharide ATP1-1 could not be separated, thus, nobody knows whether the ATP1-1 separated from the polysaccharide of the Acanthopanax trifoliatus has a positive effect on diabetes. The invention with the application number CN2016110462538 discloses a method for separating and purifying Acanthopanax trifoliatus polysaccharide to obtain neutral and uniform polysaccharide ATP1-1, which solves the problems of insufficient separation and purification of the existing Acanthopanax trifoliatus polysaccharides and low purity, providing a research basis for the present invention.

### SUMMARY

The purpose of the present invention is to provide a new way for the existing technology of preparing medicines for treatment of diabetes, specifically, it provides use of Acanthopanax trifoliatus polysaccharide ATP1-1 in preparation of drugs for treatment of diabetes.

For achieving this objective, the present invention adopts the following technical solutions:

The Acanthopanax trifoliatus polysaccharide ATP1-1 is used in preparation of drugs for treatment of diabetes.

In application, the Acanthopanax trifoliatus polysaccharide is combined with pharmaceutically acceptable excipients to prepare any pharmaceutically acceptable dosage form.

An extraction process of the Acanthopanax trifoliatus polysaccharide ATP1-1 of the present invention includes: by water extraction and alcohol precipitation, the crude polysaccharide ATP (Acanthopanax trifoliatus polysaccharide) is prepared from the stems of Acanthopanax trifoliatus, the neutral polysaccharide ATP1 and the acidic polysaccharose ATP2 and ATP3 are separated by DEAE-52 cellulose column chromatography. ATP1 undergoes repeatedly Sephadex G-75 dextran gel column chromatography to obtain neutral polysaccharide ATP1-1 with uniform molecular weight. For details, please refer to the invention with the application number CN2016110462538, which relates to a method for separating and purifying Acanthopanax trifoliatus polysaccharide.

In the present invention, raw materials of the Acanthopanax trifoliatus polysaccharide are selected form at least one of roots, root barks, stems and leaves of a plant Acanthopanax trifoliatus (Linn.) Merr., which belongs to Acanthopanax of Araliaceae.

Preferably, the raw materials of the Acanthopanax trifoliatus polysaccharide are stems of the plant Acanthopanax trifoliatus (Linn.) Merr., which belongs to Acanthopanax of Araliaceae. Since the Acanthopanax trifoliatus has the highest polysaccharide content in its stem, the polysaccharide is preferably extracted from the stems of the Acanthopanax trifoliatus.

Correspondingly, the present invention puts forward the application of Acanthopanax trifoliatus stem polysaccharide in preparation of drugs for treatment of diabetes, wherein the Acanthopanax trifoliatus stem polysaccharide is a polysaccharide extracted from the stems of the plant Acanthopanax trifoliatus (Linn.) Merr., which belongs to Acanthopanax of Araliaceae.

The beneficial effects of the above technical solutions of the present invention are as follows:

Through experiments, the present invention demonstrates that the Acanthopanax trifoliatus polysaccharide ATP1-1 can alleviate the symptoms of weight loss in diabetic mice and facilitate the weight increase of mice; ATP1-1 has a hypoglycemic effect on the diabetic mice, and can enhance the ability to control blood glucose, reduce blood glucose fluctuation, and achieve the effect of treating diabetes; various dosages of ATP1-1 can effectively reverse the decrease of insulin caused by injury of pancreatic islet, so as to achieve the effect of treating diabetes; a high or medium dose of ATP1-1 has various degrees of therapeutic effects on mice pancreatic islets and can inhibit apoptosis of pancreatic islet cells; ATP1-1 can effectively repair injury of a spleen and regulate the body's immunologic function. Further, the Acanthopanax trifoliatus is a pure natural plant, which can be developed and utilized to reduce the side effects of western medicine; and the Acanthopanax trifoliatus has the highest polysaccharide content in its stem, which has the advantage of low price. Therefore, the development of the Acanthopanax trifoliatus with hypoglycemic effect has extremely high economic value. In conclusion, the Acanthopanax trifoliatus polysaccharide ATP1-1 provided by the present invention can effectively reduce the fasting blood glucose of STZ model mice and regulate the body's immunity.

### BRIEF DESCRIPTION OF THE DRAWINGS

FIG. 1A shows the effect of ATP1-1 on the diet consumption of diabetic mice (x̅±s, n=8), compared with Week 0's high glucose group: ^{#}P<0.05, ^{##}P<0.01; compared with Week 6's high glucose group: *P<0.05, **P<0.01;

FIG. 1B shows the effect of ATP1-1 on the water consumption of diabetic mice (x̅±s, n=8), compared with Week 0's high glucose group: ^{#}P<0.05, ^{##}P<0.01; compared with Week 6's high glucose group: *P<0.05, **P<0.01;

FIG. 2 shows the effect of ATP1-1 on fasting blood glucose in diabetic mice (x̅±s, n=8), compared with high glucose group: *P<0.05, **P<0.01;

FIG. 3A shows the effect of ATP1-1 on mice glucose tolerance (x̅±s, n=8), demonstrated in the blood glucose-time curves, compared with high glucose group: *P<0.05; **P<0.01; compared with the normal group: ^{#}P<0.05; ^{##}P<0.01;

FIG. 3B shows the effect of ATP1-1 on mice glucose tolerance (x̅±s, n=8), demonstrated in the blood glucose AUC value of mice in each group, compared with high glucose group: *P<0.05; **P<0.01; compared with the normal group: ^{#}P<0.05; ^{##}P<0.01;

FIG. 4 shows the effect of ATP1-1 on mice serum insulin (x̅±s, n=8), compared with high glucose group: *P<0.05; **P<0.01; compared with the normal group: ^{#}P<0.05; ^{##}P<0.01;

FIG. 5 shows the effect of ATP1-1 on the pathological changes of mice pancreas under an electron microscope (400×);

FIG. 6 shows the effect of ATP1-1 on the pathological changes of mice pancreas under an electron microscope (200×);

FIG. 7A shows the effect of ATP1-1 on expression quantity of PPARγ in mice (x̅±s, n=8), in terms of mRNA expression quantity; compared with high glucose group: *P<0.05; **P<0.01; compared with the normal group: ^{#}P<0.05; ^{##}P<0.01;

FIG. 7B shows the Western Blot results demonstrating the effect of ATP1-1 on expression quantity of PPARγ in mice (x̅±s, n=8), in terms of PPARγ protein expression quantity; compared with high glucose group: *P<0.05; **P<0.01; compared with the normal group: ^{#}P<0.05; ^{##}P<0.01;

FIG. 7C shows the effect of ATP1-1 on expression quantity of PPARγ in mice (x̅±s, n=8), in terms of the PPARγ protein expression quantity; compared with high glucose group: *P<0.05; **P<0.01; compared with the normal group: ^{#}P<0.05; ^{##}P<0.01;

FIG. 8A shows the effect of ATP1-1 on mice spleen cytokines (x̅±s, n=8), in terms of IFN-γ quantity, compared with high glucose group: *P<0.05; **P<0.01; compared with the normal group: ^{#}P<0.05; ^{##}P<0.01;

FIG. 8B shows the effect of ATP1-1 on mice spleen cytokines (x̅±s, n=8), in terms of IL-10 quantity, compared with high glucose group: *P<0.05; **P<0.01; compared with the normal group: ^{#}P<0.05; ^{##}P<0.01.

### DETAILED DESCRIPTION OF THE EMBODIMENTS

In order to make the technical problems to be solved by the present invention, technical solutions and advantages more explicit, the following will describe in detail with reference to specific embodiments.

By water extraction and alcohol precipitation, the crude polysaccharide ATP (Acanthopanax trifoliatus polysaccharide) is prepared from the stems of Acanthopanax trifoliatus, the neutral polysaccharide ATP1 and the acidic polysaccharose ATP2 and ATP3 are separated by DEAE-52 cellulose column chromatography. ATP1 undergoes repeatedly Sephadex G-75 dextran gel column chromatography to obtain neutral polysaccharide ATP1-1 with uniform molecular weight, in order to study the immune regulation mechanism of ATP1-1 in the treatment of diabetic mice.

### 1. METHOD ESTABLISHMENT OF DIABETES MICE MODEL

After 70 mice were adaptively fed for 3 days, they were fasted and kept feeding water for 12 hours, 0.1 mol/L STZ-streptozotocin solution was injected intraperitoneally, the injection volume was 60 mg/kg, and the injection was continued for 5 days. Seven days after the injection, the mice were fasted and kept feeding water for 8 hours, and the tail was cut to collect blood and the fasting blood glucose of the mice was measured. The model was considered as a successful model with a blood glucose concentration greater than 16.5 mmol/L. Diabetic mice successfully modeled (50) were randomly divided into 5 groups, namely: a high glucose model group, a dimethylbiguanid group (185 mg/kg), ATP1-1 high dose group (140 mg/kg), ATP1-1 medium dose group (70 mg/kg), ATP1-1 low dose group (35 mg/kg), 10 mice in each group, and 10 normal mice were randomly selected as a normal control group. All groups were administrated by intragastric administration, and the normal control group and high glucose model group were administrated the same amount of distilled water. after the successful modelling, the body weight, diet and water consumption and blood glucose of the mice before administration and 1, 2, 3, 4, 5, and 6 weeks after administration were measured, and the glucose tolerance was measured at the end of the experiment.

4 weeks after administration, the mice were fasted and kept feeding water for 10 hours. All mice were sacrificed, dissecting the mouse to obtain each mouse organ; the pancreas and spleen tissues were fixed with formalin for sectioning; the rest were stored in liquid nitrogen for use.

### 2. RESULTS

2.1 the Effect of ATP1-1 on the Body Weight of Mice

After modelling, the body weight of STZ-induced diabetic mice decreased significantly. As shown in Table 1, before administration, there is no difference in body weight of each model mouse (P>0.05), the body weight of the normal control group was significantly higher than the other groups (P<0.01). During the administration period, the body weight of the dimethylbiguanide group, ATP1-1 high dose group and medium dose group all increased. After 6 weeks of administration, the body weight of the mice in these three groups was significantly higher than that of the high glucose model group (P<0.05). It shows that a high or medium dose of ATP1-1 can relieve the symptoms of weight loss in diabetic mice and facilitate the weight increase of mice.

TABLE 1
Effect of ATP1-1 on the body weight of mice ( x±s, n=8)
Body Weight (g)

| | | | Dimethylbig | | ATP1-1 | |
|---|---|---|---|---|---|---|
| | Normal | High glucose | uanide | ATP1-1 high | medium dose | ATP1-1 low |
| | group | group | group | dose group | group | dose group |
| Week 0 | 21.78± .56* | 19.31±.88^{#} | 19.48±.78^{#} | 19.43±.84^{#} | 19.77±.66^{#} | 19.70±.56^{#} |
| Week 1 | 21.93±.56* | 19.03±.87^{#} | 19.72±.77 | 20.05±.84 | 19.77±.72 | 19.63±.57^{#} |
| Week 2 | 22.05±.56* | 18.78±.90^{#} | 20.40±.78 | 20.51±.88 | 19.93±.79 | 19.94±.54^{#} |
| Week 3 | 22.62±.54** | 18.71±.87^{##} | 20.94±.73 | 20.90±.84 | 20.06±.79^{#} | 20.12±.71^{##} |
| Week 4 | 23.07±.47** | 18.69±.85^{##} | 21.49±.84* | 21.64±.95* | 20.73±.92 | 20.50±.57^{##} |
| Week 5 | 23.88±.46** | 18.81±.82^{##} | 21.70±.90* | 21.93±1.03* | 21.25±.88^{#} | 20.96±.60^{##} |
| Week 6 | 24.09±.42** | 18.79±.81^{##} | 21.89±.89* | 22.06±1.10* | 21.63±.90*^{#} | 21.15±.86^{#} |

Note: ^{#}13 <0.05;
^{##}413 <0.01 compared with the normal group;
*P <0.05;
**P <0.01 compared with high glucose group

2.2 the Effect of ATP1-1 on the Diet and Water Consumption of Mice

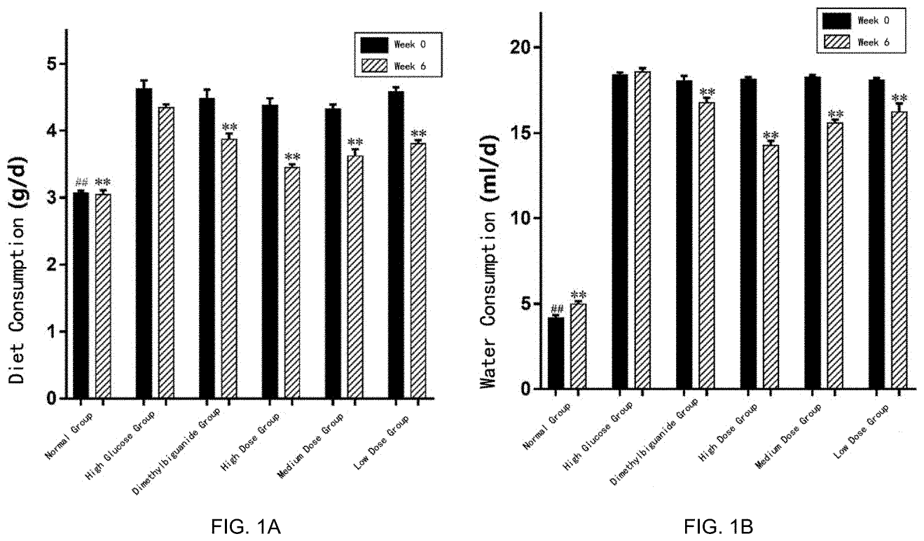

The typical features of diabetes are increased diet and drinking water. As shown in FIGS. 1A-B, before administration, the diet and water consumption of diabetic mice was significantly higher than that of normal control mice (P<0.01). During the administration period, compared with the high glucose model group, the diet and water consumption in the dimethylbiguanide group, the ATP1-1 high, medium, and low dose groups were significantly reduced (P<0.01), which demonstrates that all doses of ATP1-1 can alleviate the symptoms of polyphagia in diabetic mice.

2.3 the Effect of ATP1-1 on the Fasting Blood Glucose of Mice

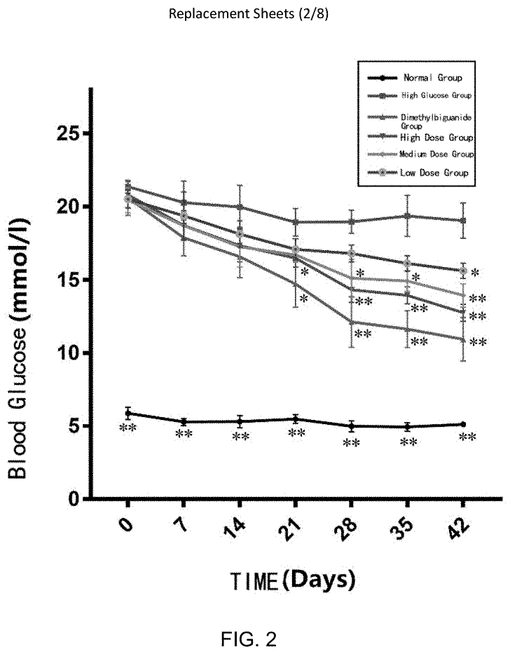

As shown in FIG. 2, the fasting blood glucose values of each group of mice before administration were all >16.5 mmol·L⁻¹, and the blood glucose of the normal control group was significantly different from that of the high glucose group (P<0.01); there was no significant difference between each administration group and the high glucose model group (P>0.05), and the modelling was successful. The blood glucose of the high glucose group remained stable during 6 weeks of administration, in contrast, after three weeks of administration, the blood sugar of the dimethylbiguanide group and the ATP1-1 high dose group was significantly different from that of the high glucose group (P<0.05). During 6 weeks of high dose administration of ATP1-1, the blood glucose of diabetic mice can be continuously and greatly reduced. At the end of the administration, the blood glucose inhibition rate of the dimethylbiguanide group and the ATP1-1 high, medium, and low dose groups were 46.9%, 38.9%, 31.8%, and 23.8%, respectively. It can be seen that each dose of ATP1-1 has a better hypoglycemic effect on diabetic mice, and within the experimental range, the effect is dose-dependent, to some degree.

2.4 the Effect of ATP1-1 on Glucose Tolerance of Mice

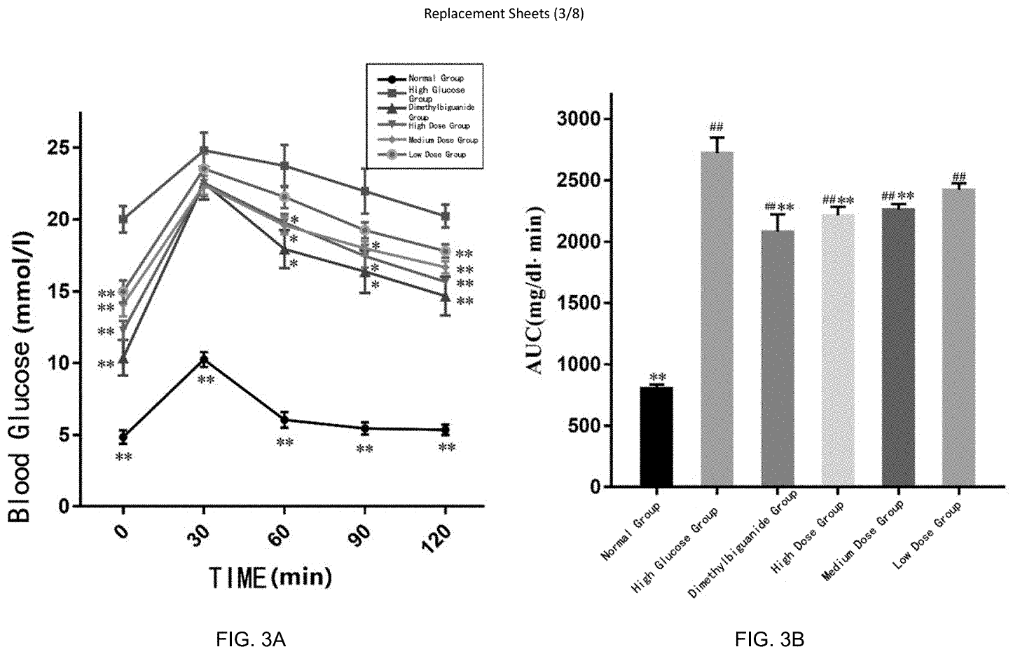

As shown in FIG. 3A, medium and high doses of ATP1-1 can effectively reduce the peak of blood glucose produced by mice after taking glucose, and can accelerate the return of blood glucose to normal value, indicating that ATP1-1 can enhance the ability to control blood glucose, reduce blood glucose fluctuation, and achieve the effect of treating diabetes. At the same time, as shown in FIG. 3B, compared with the high glucose group, the glucose tolerance of each administration group was significantly reduced, further indicating that ATP1-1 can enhance the body's control of blood glucose and treat diabetes in model mice.

2.5 the Effect of ATP1-1 on Serum Insulin of Mice

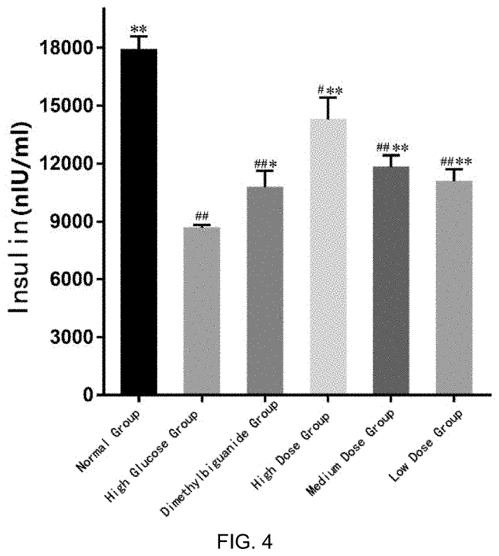

The results in FIG. 4 show that the serum insulin content of the successfully modeled mice is greatly reduced, after 6 weeks of treatment, the insulin content of mice in each dose group of ATP1-1 increased significantly, indicating that various dosages of ATP1-1 can effectively reverse the decrease of insulin caused by injury of pancreatic islet, so as to achieve the effect of treating diabetes.

2.6 the Effect of ATP1-1 on the Pathological Changes of Pancreas and Spleen

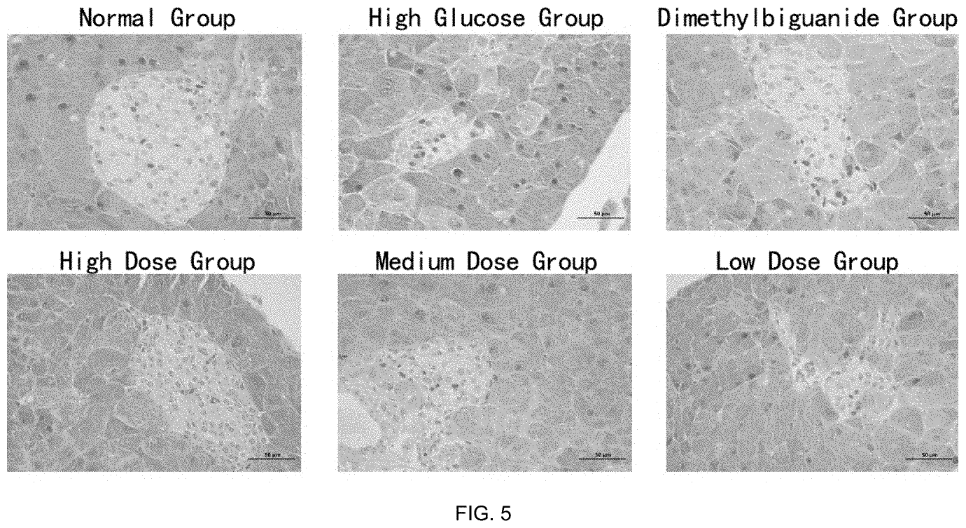

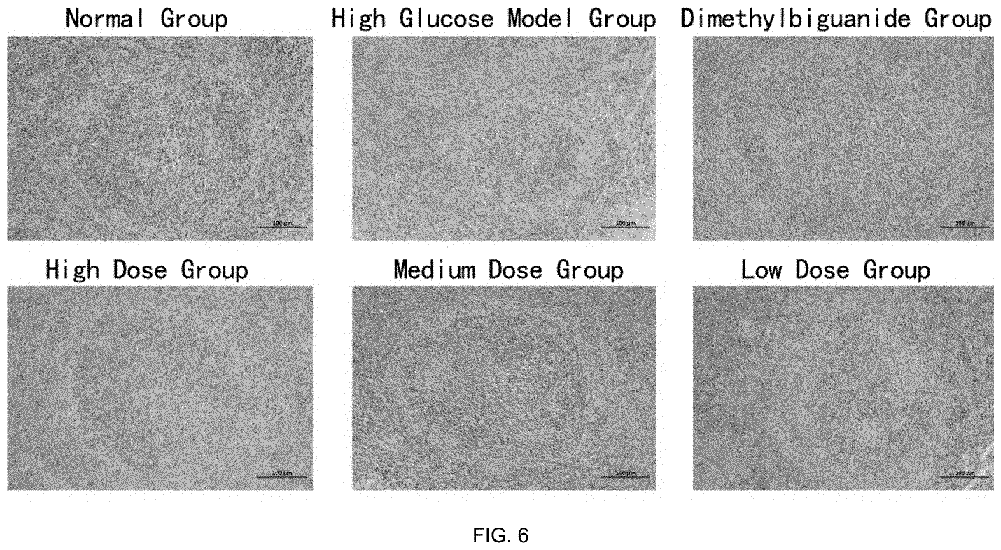

As shown in FIGS. 5 and 6, at the end of the experiment, the spleens and pancreas of each group of mice were taken for HE staining, morphology observation results of pancreatic tissues showed that normal pancreatic islets were round, with regular cell distribution, rich and lightly stained cytosol, wherein pancreatic islets were distributed between acinuses, and had a clear boundary with the exocrine glands; the pancreatic islets in the high glucose model group were irregular in shape, and the boundary with the exocrine glands was not clear, the pancreatic islet endocrine cells were unevenly distributed, the pancreatic islet cells were swollen, and cell vacuolar degeneration appeared. In the ATP low dose group, pancreatic islets showed irregular morphology under the microscope, and the boundary was not clear, and there were still many islet cells with vacuolar degeneration; after treated with a medium dose of ATP, it was found that mice pancreatic islets are more regular and rounder, with relatively clear boundary and regular cell distribution, but there was still cell vacuoles degeneration. Observation of mice in the ATP high dose group showed that the islets of the present group were regular and round in shape, and most of the islet cells were regular, with round cell nucleus and rich cytosol. These showed that the high or medium dose of ATP1-1 has various degrees of therapeutic effects on mice pancreatic islets and can inhibit apoptosis of pancreatic islet cells.

FIG. 6 showed that compared with the normal group, the volume of red pulp in the spleen of mice in the high glucose group increased, and the white pulp decreased; ATP1-1 treatment can reverse this injury, and the spleen cells of the mice in the high dose group were arranged tightly and orderly, most similar to the normal group, indicating that ATP1-1 can effectively repair injury of a spleen and regulate the body's immunologic function.

2.7 the Effect of ATP1-1 on PPARγ Expression Quantity

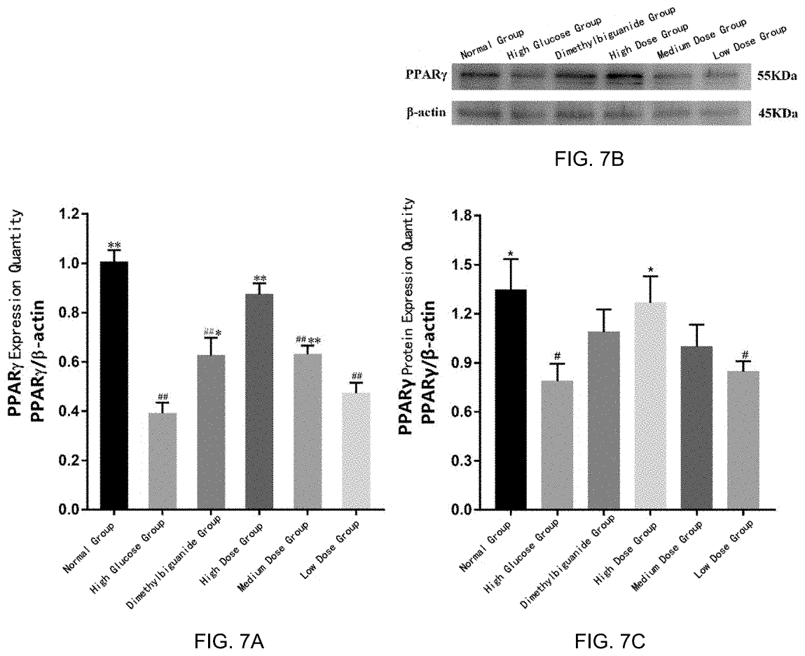

In the experiment, qPCR and Western Blot methods were used to study the expression of PPARγ at the gene and protein level, and results were shown in FIGS. 7A-C. The results showed that, at the gene and protein level, the expression quantity of PPARγ in STZ-induced diabetic mice decreased significantly. Treatment with ATP1-1 for 6 weeks can effectively increase the expression quantity of PPARγ. PPARγ is a member of the nuclear hormone receptors and is closely involved in regulating the expression of a variety of genes, so as to regulate the metabolism, differentiation and apoptosis of cells, and there is an interaction between it and the cytokines Th1 and Th2, the increase of PPARγ can decrease the cytokine Th1 and increase Th2, thereby regulating immunity and effectively lowering blood glucose.

2.8 the Effect of ATP1-1 on Cytokines

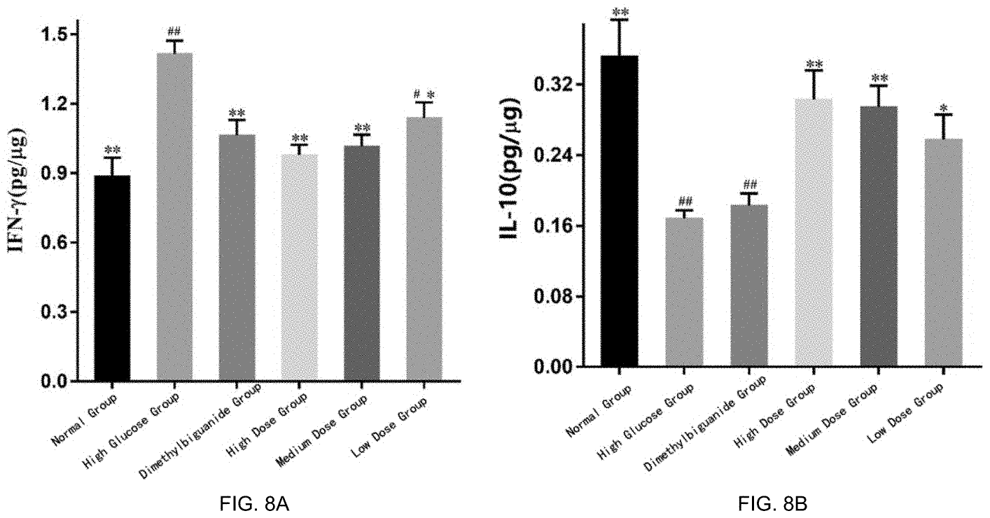

As shown in FIGS. 8A-B, in the diabetic state, IL-10 of mice was significantly reduced, and IFN-γ was significantly increased. ATP1-1 treatment can reverse this change, IFN-γ and IL-10 are cytokines secreted by Th1 and Th2 cells, respectively; the content of the two cytokines can represent the ratio of Th1/Th2 cells. Th1/Th2 imbalance can induce diabetes, and the results suggested that ATP1-1 treatment can adjust the balance of Th1/Th2, regulate the body's immunity, and improve diabetes.

### 3. CONCLUSION

ATP1-1 can effectively reduce the fasting blood glucose of STZ model mice and regulate the body's immunity, so that ATP1-1 can be used to prepare medicines for treating diabetes.

Specific embodiments of the present invention have been described above, but the specific embodiments are only used as examples and should not be taken as limitation. Those skilled in the art should be able to understand that any equivalent modifications and substitutions to the present invention are within the scope of the present invention. Therefore, without departing from the spirit and the scope of the present invention, a variety of equivalent changes and modifications should be within the scope of the present invention.

What is claimed is:

1. A method of treating diabetes, comprising the step of preparing Acanthopanax trifoliatus polysaccharide ATP1-1 into drugs for treating the diabetes.

2. The method according to claim 1, wherein raw materials of the Acanthopanax trifoliatus polysaccharide ATP1-1 are selected from at least one of roots, root barks, stems and leaves of a plant of Acanthopanax trifoliatus (Linn.) Merr., wherein the Acanthopanax trifoliatus (Linn.) Merr. belongs to Acanthopanax of Arahaceae.

3. The method according to claim 2, wherein the raw materials of the Acanthopanax trifoliatus polysaccharide ATP1-1 are the stems of the plant of the Acanthopanax trifoliatus (Linn.) Merr.

4. (canceled)
